(19) United States
(12) Patent Application Publication
(10) Pub. No.: US 2021/0008329 A1
(43) Pub. Date: Jan. 14, 2021

(54) SLEEP PARALYSIS DETECTION DEVICE WITH EXTERNAL STIMULATION

(71) Applicants: Mica Nakai Sloan, Salt Lake City, UT (US); Joseph B Arrington, Salt Lake City, UT (US)

(72) Inventors: Mica Nakai Sloan, Salt Lake City, UT (US); Joseph B Arrington, Salt Lake City, UT (US)

(21) Appl. No.: 15/876,137

(22) Filed: Jan. 20, 2018

(51) Int. Cl. A61M 21/00

(52) U.S. Cl. CPC A61M 21/00; A61M 2021/0083; A61M 2021/0066; A61M 2205/3303; A61M 2021/0022; A61M 2230/06; A61M 2210/083; A61M 2021/0027

(57) ABSTRACT

The present invention relates to a device for providing an intervention for users who suffer from sleep paralysis. More specifically, the present invention illustrates a wearable device that measures the user's heart rate while they sleep. If the heart rate rises above a pre-set threshold level, then the device activates an element that exerts an external stimulus on the user causing the user to wake or providing a focal point for the user to overcome the sleep paralysis.

### I. BACKGROUND

Sleep paralysis is a condition that afflicts people across the world, even though many cultures justify it in different ways. It is frequently manifested when the person suffering the condition is fully conscious but unable to move their body. An attack is frequently accompanied by hallucinations, which leads to many cultures blaming the condition to super-natural beings.

The subject matter claimed herein is not limited to embodiments that may be used to treat the sleep paralysis condition. Rather, this is only an illustrative embodiment where the technology may be practiced.

### II. BRIEF SUMMARY OF THE INVENTION

This summary of the present invention is not intended to describe each illustrated embodiment or every possible implementation of the present invention. The figures and the detailed description that follow, however, do particularly exemplify these embodiments. The present invention, which relates to a device that performs an intervention on a person that is experiencing a sleep paralysis episode. In various embodiments, the device is configured to house a biometric sensor that can be worn near the user's skin during sleep. For example, in one embodiment that biometric sensor may be a heart rate monitor. The biometric sensor measures the user's vital signs to determine whether the person is experiencing a sleep paralysis episode. For example, in the previously mentioned embodiment, the heart rate monitor may detect an elevated heart rate as a result of the sleep paralysis episode.

Additionally, in various embodiments, if the biometric sensor measures a change in vital signs that may indicate a sleep paralysis episode, then a signal may be sent to an element for inducing an external stimulus. In one embodiment that may be a vibrator to induce a physical stimulus. In another embodiment, the element may be a speaker to induce an auditory stimulus.

Additional features and advantages of the invention will be set forth in the description which follows, and in part will be obvious from the description, or may be learned by the practice of the invention. The features and advantages of the invention may be realized and obtained by means of the instruments and combinations particularly pointed out in the appended claims. These and other features of the present invention will become more fully apparent from the following description and appended claims, or may be learned by the practice of the invention as set forth hereinafter.

### III. BRIEF DESCRIPTION OF THE SEVERAL VIEWS OF THE DRAWINGS

In consideration of the following detailed description of various embodiments, the invention may be more completely understood in connection with the following drawings.

FIG. 1 illustrates a side view of one embodiment of the device.

FIG. 2 illustrates a front view of one embodiment of the device.

FIG. 3 illustrates a top view of one embodiment of the device.

Although the invention is amenable to various modifications and alternative forms, specifics thereof have been shown by way of example in the drawings and will be described in detail such that the disclosure will be thorough and complete, and will fully convey the scope of the invention to those skilled in the art. However, it should be understood, that the intention is not to limit the invention to the particular embodiments described. On the contrary, the intention is to cover all modifications, equivalents, and alternatives falling within the spirit and scope of the invention as defined by the appended claims.

### IV. DETAILED DESCRIPTION OF THE INVENTION

Sleep Paralysis is an episodic condition where the patient's typical sleep pattern is interrupted. Although, many people suffer these symptoms only a few times during their life, chronic suffers may experience them many times a year. Typically, during sleep brain activity decreases until the person enters rapid eye movement (“REM”) sleep. At that stage, brain activity increases while dreaming but the body enters a state of paralysis. However, during a sleep paralysis episode, the person does not achieve REM sleep, instead the person gains consciousness but the body is paralyzed. Thus, the person is conscious of their state, but unable to move their body or to wake up. This condition is frequently accompanied by hallucinations that in many cultures have become folkloric, where often the sleeper is “attacked” by a supernatural being that is restricting their movement.

Currently, Among the devices on the market is the Sleep Guardian. However, this device is targeted specifically for Night Terrors experienced by small children and not for children and adults suffering from Sleep Paralysis. The Sleep Guardian is placed underneath the mattress and is turned on. When the device detects movement, it begins to vibrate to soothe the child and prevent them from waking. The device's primary purpose is to help the child remain asleep and overcome night terrors through time.

However, sleep paralysis differs from Night Terrors in that some people do not grow out of them and it becomes part of their everyday life. Further, a person experiencing a sleep paralysis episode may not activate the Sleep Guardian because they are paralyzed and thus do not have the requisite movement to activate the vibration. Rather, an episode of sleep paralysis may be identified by an increase in heart rate due the stress of the person as they realize that they cannot move. Thus, people that experience sleep paralysis are limited to confronting this condition on their own or relying on other people to identify an episode and provide external stimulation to either wake or provide a focal point for the person to overcome the episode.

The lack of interventions prevents people who suffer regular sleep paralysis episodes from living a stereotypical life. For example, the individual may be limited in his/her ability to travel for work because of the inability to overcome a sleep paralysis episode. Often time, people that suffer from regular episodes are afraid to go to sleep, or even may turn to alcohol to escape the fear. However, this may exacerbate the problem because the person is unable to reach delta stage sleep—the same result as a sleep paralysis.

In addition to helping people suffering from sleep paralysis, the device may also be used to help people suffering from night terrors, PTSD, and chronic nightmares. Although currently available devices use motion to trigger the intervention, night terrors, PTSD, and chronic nightmares could be served by the same device that treats sleep paralysis.

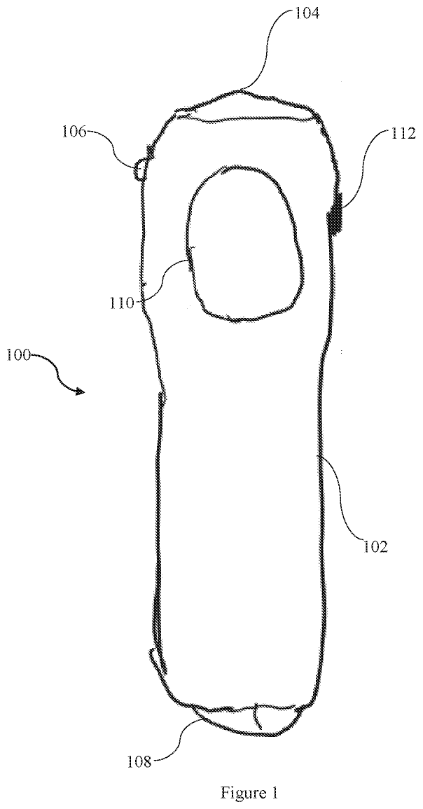

FIG. 1 illustrates a side view of one embodiment of the device 100. The device 100 may include a flexible housing 102, a display 104, a switch 106, a clasp 108, rumble packs 110, and a data/power interface port 112, according to some embodiments. In one embodiment, the switch 106 may be located near the display 104 and positioned on the flexible housing 102. The clasp 108 may also be placed on the flexible housing. The flexible housing 102 may also support the rumble packs 110. The flexible housing may further include an aperture for the data/power interface port 112.

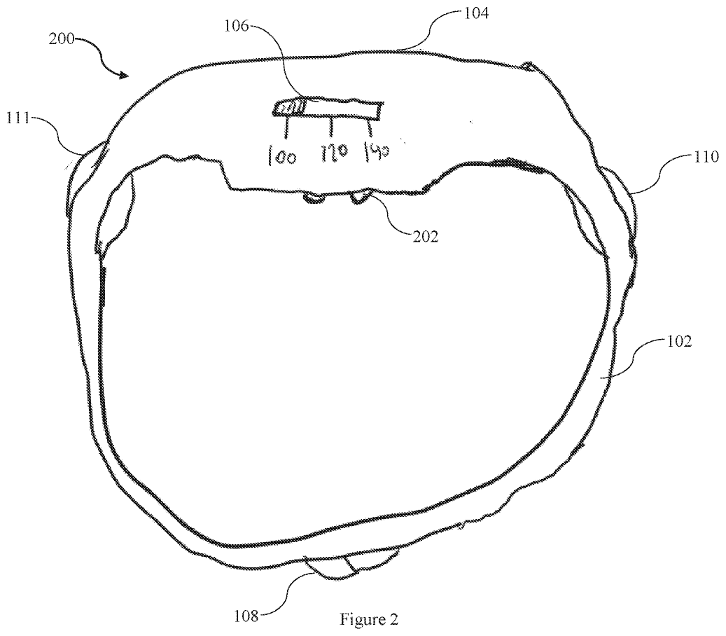

FIG. 2 illustrates a front view of one embodiment of the device 200. The device 200 may include a flexible housing 102, a display 104, a switch 106, a clasp 108, a first rumble pack 110, a second rumble pack 111, and a biometric sensor 202. The flexible housing 102 may be used to secure the switch 106, the clasp 108, the first rumble pack 110, and the second rumble pack 111 in a fixed position, in some embodiments. In another embodiment, the biometric sensor 202 may also be secured in a fixed position using the flexible housing 102.

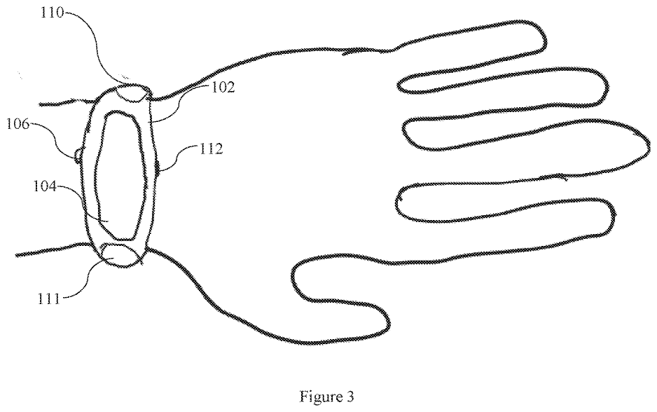

FIG. 3 illustrates a top view of one embodiment of the device as it would be configured when worn on the wrist of a user. In one embodiment, the device may include a flexible housing 102, a display 104, a switch 106, a first rumble pack 110, a second rumble pack 111, and a data/power interface port 112. The switch 106 may be accessible while worn with the first rumble pack 110 configured to be placed over the ulna of the user and the second rumble pack 111 configured to be placed over the radius of the user. In other embodiments, the data/power interface port may be housed within the flexible housing 102.

The present invention relates to a device for providing intervention for sleep paralysis episodes. FIGS. 1, 2, and 3 show an illustrative embodiment of the invention. More specifically, in one embodiment of the invention, an elongated flexible housing 102 may be formed out of a flexible yet sturdy material, for example, a bonded polymer substance or silicone. The flexible housing 102 may be worn by a person. For example, the ends of the housing may be wrapped around a person's wrist and closed using a clasp 108. However, the invention is not limited to use on a person's wrist, the invention may be configured to be worn anywhere that could read the person's biometric information and provide an external stimulus.

The flexible housing 102 may be configured with openings to correspond to other elements. For example, in one embodiment, a display 104 may be located on the top, near the middle, of the flexible housing 102. The display 104 may be configured to display basic information such as the time and biometric information. For example, the display may show the heart rate and blood oxygen level of the patient. However, this invention is not limited to a screen to show biometric information. In another embodiment, instead of using a display to show biometric information, that biometric information could be stored in the memory and transmitted to an external computing device, such as a computer, tablet, or smart phone, through either a wireless transmission, such as Bluetooth or over wireless network connection, or through a hard-wired, such as a micro-USB or USB-C cable.

Returning the embodiment illustrated in FIGS. 1 and 2, a switch 106 may be located near the middle of the flexible housing 102 but on the side rather than the top. However, the location of the switch 106 is not limited to the specified location, rather it may be located at any convenient position. The switch 106 may be configured to set the heart rate activation threshold. This allows the patient to set the heart rate threshold for which the other elements activate. In another embodiment, the heart rate activation threshold may be set through an application installed on an external computing device, such as a computer or smart phone. The heart rate activation threshold may be communicated to the device through either a direct wireless connection, such as Bluetooth, or through wireless network connection, or through a hard-wired connection, such as micro-USB cable or USB-C cable.

In the illustrative embodiment from FIGS. 1, 2, and 3, the user's heart rate is measured through a biometric sensor 202 located on the bottom, near the middle of the length, of the flexible housing 102. The biometric sensor 202 may be configured as to rest on the skin of the user when the device is worn by the user. The biometric sensor 202 may measure vital signs of the user which may be compared to the levels set by the switch 106.

As shown in FIGS. 1, 2, and 3, a first rumble pack 110 and a second rumble pack 111 may be placed in the flexible housing 102. The first rumble pack 110 may be configured to be placed on the ulna of the user. While, the second rumble pack 111 may be configured to be placed on the radius of the user. The first rumble pack 110 and the second rumble pack 111 may induce an external stimulus on the user. As the person's vital signs as measured by the biometric sensor 202 exceeds the threshold level set by the switch 106, or an alternative method for different embodiments discussed, the first rumble pack 110 and the second rumble pack 111 may be activated and vibrate producing an external stimulus that may either wake the person wearing this embodiment or provide a focus point to facilitate overcoming the paralysis. In another embodiment, the external stimulus may be produced by one or more speakers that produce an external stimulus that may cause the user to wake or provide a focal point for overcoming the paralysis. In yet another embodiment, an element may change temperature to provide an external stimulus that may help the wearer to wake up or to provide a focal point.

In the embodiment illustrated in FIGS. 1, 2, and 3, a data/power connector 112 is placed within the flexible housing 102. The data/power connector 112 may be a micro-USB connector or a USB-C connector through which the battery may be charged or data may be retrieved. In another embodiment, the battery may be charged using a wireless charging system and the data may be retrieved using wireless data transmission methods, such as Bluetooth or wireless network connection.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

The various components of the present invention may be constructed generally out of any materials known to be suitable in the art.

Many modifications and other embodiments of the invention set forth herein will come to mind to one skilled in the art to which this invention pertains and having the benefit of the teaching presented in the foregoing descriptions and the associated drawings. Therefore, it should be understood that the inventions are not to be limited to the specific embodiments disclosed and that modifications and other embodiments are intended to be included within the scope of the appended claims. Although specific terms are employed herein, they are used in a generic and descriptive sense only, and not for purposes of limitation.

We claim:

1. An apparatus comprising:
a biometric sensor; and
an element as a means for exerting a stimulus on the patient.

2. The apparatus as in claim 1, wherein the biometric sensor is a heart rate monitor.

3. The apparatus as in claim 1, wherein the external stimulus is an auditory stimulus, such as noise exerted from a speaker.

4. The apparatus as in claim 1, wherein the element that exerts the stimulus is a rumble pack.

5. The apparatus as in claim 4, wherein the rumble pack is enclosed in a wearable element that is worn on the patient's wrist.

6. The apparatus as in claim 5, wherein the wearable element is formed of a bonded polymer substance, such as silicone.

7. A wearable apparatus comprising:
a wearable element formed of a flexible material;
a biometric sensor, wherein the biometric sensor is mounted on the wearable element;
an element configured to induce an external stimulus mounted on the wearable element.

8. The wearable apparatus as in claim 7, wherein the biometric sensor is a heart rate monitor.

9. The wearable apparatus as in claim 7, wherein the wearable element is a wrist band.

10. The wearable apparatus as in claim 9, wherein in the wrist band is formed of a bonded polymer substance, such as silicone.

11. The wearable apparatus as in claim 7, wherein the element for inducing the external stimulus is a rumble pack.

12. The wearable apparatus as in claim 7, where in the element for inducing the external stimulus is a speaker.

13. The wearable apparatus as in claim 7, where in the element configured to induce external stimulus is an element that changes temperature.

14. A wearable device comprising:
an elongated wearable element configured to be worn on the user's wrist;
a display housed within the wearable element near middle of the wearable element configured such that when the wearable element is placed on a user's wrist the display is face up;
a switch located near the middle of the wearable element configured so that when the wearable element is placed on a user's wrist the switch is accessible;
a biometric sensor configured on the wearable element so that when a user places the wearable element on their wrist, the biometric sensor rests against the user's skin;
one or more elements as a means for exerting an external stimulus on the user;
a clasp element located on one or both of the ends of the wearable element as a means for connecting the two ends and allowing for the wearable element to be worn by the user;
a data port housed within the wearable element.

15. The apparatus as in claim 14, wherein the wearable element is formed of a bonded polymer substance, such as silicone.

16. The apparatus as in claim 14, wherein the biometric sensor is a heart rate monitor.

17. The apparatus as in claim 14, wherein the element for exerting an external stimulus is one or more rumble packs.

18. The apparatus as in claim 14, wherein the element that exerts an external stimulus is two rumble packs, one configured to be placed over the user's ulna and the second configured to be placed over the user's radius.

19. The apparatus as in claim 14, wherein the element that exerts an external stimulus is one or more speakers to produce an auditory external stimulus.

20. The apparatus as in claim 14, wherein the element that exerts an external stimulus changes in temperature to exert an external stimulus on the user.
